(19) United States
(12) Patent Application Publication
(10) Pub. No.: US 2025/0075036 A1
(43) Pub. Date: Mar. 6, 2025

(54) ARYL-BISIMIDAZOLIUM POLYMERS AND USES THEREOF

(71) Applicant: IONOMR INNOVATIONS INC., Vancouver (CA)

(72) Inventors: Bobak Gholamkhass, North Vancouver (CA); Benjamin Britton, Vancouver (CA); Wei Li, Burnaby (CA)

(73) Assignee: IONOMR INNOVATIONS INC., Vancouver (CA)

(21) Appl. No.: 18/820,653

(22) Filed: Aug. 30, 2024

Related U.S. Application Data
(60) Provisional application No. 63/580,118, filed on Sep. 1, 2023.

(51) Int. Cl. C08G 73/06; B01D 71/62

(52) U.S. Cl. CPC C08G 73/0616; B01D 71/62; B01D 2325/42

(57) ABSTRACT

The present disclosure features a poly(bis-arylimidazolium) polymer and synthesis thereof, having a controlled molecular weight and specified falling ball viscosity. The disclosed poly(bis-arylimidazolium) polymers yield desirable mechanical properties when incorporated into an ionic membrane. Ionic membranes comprising the disclosed poly(bis-arylimidazolium) polymers can be incorporated into an electrochemical device such as a fuel cell, an electrolyzer, a redox flow battery, or another electrochemical device.

### CROSS-REFERENCE(S) TO RELATED APPLICATION(S)

This application claims the benefit of U.S. Application No. 63/580,118, filed on Sep. 1, 2023, the disclosure of which is hereby incorporated by reference in its entirety.

### BACKGROUND

Alkali anion exchange membranes (AEMs) are predominantly based on quaternary ammonium groups pendent to a polymer main chain (or “backbone”). The stability of quaternary ammonium groups is often poor in highly alkaline solutions. Thus, the performance of materials comprising anion exchange membranes having quaternary ammonium groups rapidly degrades over time.

Molecular weight (MW) and molecular weight distribution (MWD) can markedly affect the mechanical properties of a polymer. For many mechanical properties and polymers, this effect reaches a limiting value at a relatively high MW where no appreciable change on mechanical properties exists with increasing MW. Such mechanical properties include strength and toughness.

Stress-strain tests give an indication of strength and toughness of a polymer. Since toughness is the energy a material can absorb before breaking, a correlation exists between impact strength and toughness. Brittle materials show low toughness, whereas ductile materials which can be cold drawn are very tough, due to a large elongation at the break (EB).

Ultimate tensile strength (UTS) increases as MW increases in polymers such as poly (vinyl chloride) (PVC). Higher MW materials have a higher elongation to fracture, implying a larger strain hardening capacity. Further, the greater the elongation a given specimen can withstand, the greater the degree of orientation in that specimen before failure. Therefore, higher MW specimens elongate further, are more highly oriented, and exhibit a higher UTS.

Alkaline electrolytic membranes made using high molecular weight poly(bis-arylimidazolium) polymers offer a balanced alkaline stability and high ion exchange capacity (IEC). However, due to the rigid main-chain molecular structure of higher molecular weight poly(bis-arylimidazoliums), the membrane tends to become less flexible, and even brittle. The mechanical properties of such polymers are unsuitable for many applications.

Accordingly, there is a need for positively charged polymers that offer improved alkali-stability and suitable mechanical and flexibility properties, balanced with a high IEC, low water uptake, and high chemical stability.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure features a polymer having a repeating unit of Formula (I):

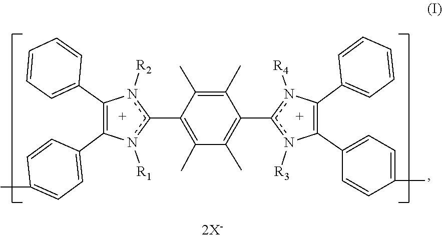

wherein:
R₁, R₂, R₃, and R₄ are each independently C₁₋₈ alkyl; and
X⁻ is a monovalent anion selected from the group consisting of F⁻, Cl⁻, Br⁻, I⁻, HO⁻, BF₄⁻, PF₆⁻, HCO₃⁻, NO₃⁻, and CH₃CO₂⁻, and
wherein the falling ball viscosity of 1 wt % polymer solution in dimethyl sulfoxide (DMSO) is from about 7 cP to about 30 cP.

In another aspect, the present disclosure features a method of making a polymer, comprising:
forming a monomeric base unit of Formula (IV);
alkylating a monomeric base unit nitrogen to form a dialkylated base unit;
forming Ni(COD)₂ in situ for polymerizing the dialkylated base unit;
polymerizing the dialkylated base unit to form a polymerized dialkylated base unit; and
alkylating a polymerized dialkylated base unit nitrogen to form a tetraalkylated polymerized base unit comprising a repeating unit of Formula (I),
wherein Formula (IV) is:

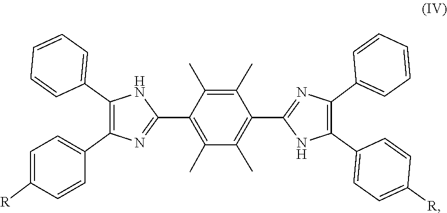

wherein R is Cl, Br, or I.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows a plot of the relationship between viscosity of the polymers, measured by falling ball viscosity of 1 wt % polymer solution in dimethyl sulfoxide (DMSO), and their molecular weight (MW), as measured by gel permeation chromatography (GPC);

FIG. 2 shows elongation at break ratio (strain %) as a function of polymer viscosity (i.e., mol. wt.) with desired range for the viscosity (10-25 cP), and accordingly MW of the polymer, for the polymer of Structure (I) wherein R₁, R₂, R₃, and R₄ are each methyl; and

FIG. 3 shows a plot of elongation at break ratio (strain %) as a function of polymer viscosity (i.e., mol. wt.) depicting desired range for the viscosity (10-30 cP), and accordingly MW of the polymer of Structure (I) wherein R₁ and R₃ are methyl, R₂ and R₄ are n-butyl, and X⁻ is chloride.

### DETAILED DESCRIPTION

The mechanical properties of polymers, and membranes formed therewith, are tunable for end-use applications by controlling the molecular weight of the poly(bis-arylimidazolium) polymers disclosed herein.

### Definitions

The terminology used in the description of the invention herein is for the purpose of describing particular embodiments, and is not intended to be limiting for the invention.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

Substituents of polymers of the disclosure are disclosed herein in groups or in ranges. It is specifically intended that the disclosure include each and every individual subcombination of the members of such groups and ranges. For example, the term “C₁₋₈ alkyl” is specifically intended to individually disclose (without limitation) methyl, ethyl, C₃ alkyl, C₄ alkyl, C₅ alkyl, C₆ alkyl, C₇ alkyl, and C₈ alkyl, whether linear or branched. For example, C₄ alkyl can be n-butyl, sec-butyl, isobutyl, or tert-butyl.

As used herein, the term “alkyl” refers to straight or branched hydrocarbon groups. In some embodiments, alkyl has 1 to 8 carbon atoms, 1 to 7 carbon atoms, 1 to 6 carbon atoms, 1 to 5 carbon atoms, 1 to 4 carbon atoms, 1 to 3 carbon atoms, 1 or 2 carbon atoms, or 1 carbon atom. Representative alkyl groups include methyl, ethyl, propyl (e.g., n-propyl, isopropyl), butyl (e.g., n-butyl, sec-butyl, isobutyl, and tert-butyl), pentyl (e.g., n-pentyl, tert-pentyl, neopentyl, isopentyl, pentan-2-yl, pentan-3-yl), hexyl (e.g., n-hexyl, geometric isomers), and octyl (e.g., n-octyl, geometric isomers) groups.

As used herein, the term “halogen,” “halo,” or “halide” refers to fluoro, chloro, bromo, and iodo groups. “Halogen,” “halo,” or “halide” can refer to the entire set of fluoro, chloro, bromo, and iodo groups, or to a subset of halogens (e.g. fluoro, chloro, and bromo; chloro, and iodo; chloro) or to any other combination or subcombination of halogen atoms.

As used herein, the term “repeating unit” corresponds to the smallest monomeric unit of a polymer, the repetition of which constitutes a macromolecule. The monomeric unit of a polymer refers to a group of atoms in a monomer, comprising a part of the polymer chain, together with its pendant atoms or groups of atoms. The monomeric unit is a repeating unit within a chain. The monomeric unit can also refer to an end group on a polymer chain. For example, the monomeric unit of polyethylene glycol can be —CH₂CH₂O— corresponding to a repeating unit, or —CH₂CH₂OH corresponding to an end group.

As used herein, the term “end group” refers to a repeating unit, or monomeric unit, with only one attachment to a polymer chain, located at the end of a polymer. For example, the end group can be derived from a monomeric unit at the end of the polymer, once the monomeric unit has been polymerized. As another example, the end group can be a part of a chain transfer agent or initiating agent that was used to synthesize the polymer.

As used herein, the term “cationic” refers to a moiety that is positively charged, or ionizable to a positively charged moiety under chemical conditions. Examples of cationic moieties include, for example, ammonium, iminium, imidazolium, oxazolium, thiazolium groups, etc.

As used herein, the term “anionic” refers to a functional group that is negatively charged, or ionizable to a negatively charged moiety under chemical conditions. Examples of anionic groups include halide, carboxylate, hydroxide, etc.

A weight percent (wt %) of a component, unless specifically stated to the contrary, is based on the total weight of the formulation or composition in which the component is included.

As used herein, “molecular weight,” or “MW,” refers to number-average molecular weight which can be measured by ¹H NMR spectroscopy, gel permeation chromatography (GPC), viscosity, falling ball viscosity, or other analytical methods. Differences in molecular weights of a polymer synthesized by the same method may be estimated by comparing viscosity of polymer solutions.

As used in the description of the invention and the appended claims, the singular forms “a”, “an” and “the” are intended to include the plural forms as well, unless the context clearly indicates otherwise.

As used herein, the term “about” can be understood to include values within 10% of the stated value. For example, a viscosity of about 7 cP means a viscosity of 7±0.7 cP, or a viscosity of 6.3-7.7 cP.

It is further intended that the compounds of the disclosure are stable. As used herein, “stable” refers to a compound that is sufficiently robust to survive isolation to a useful degree of purity from a reaction mixture.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art, and should not be interpreted in an idealized or overly formal sense unless expressly so defined herein.

### Polymers

A family of polymers capable of forming membranes having high HO⁻ ionic conductivity and caustic stability, as well as desirable mechanical properties, is presented. In some embodiments, the anion exchange membranes (AEMs) are composed of poly(aryl-bisimidazolium) backbones with N-alkyl side chains.

In one aspect, a polymer is provided. The polymer includes one or more repeating units, or monomeric units. In some embodiments, at least one repeating unit comprises Formula (I):

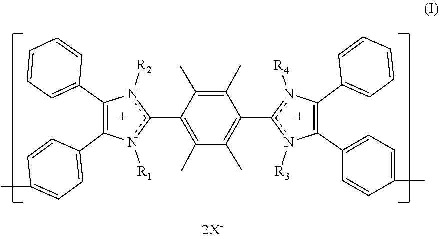

wherein:
R₁, R₂, R₃, and R₄ are each independently C₁₋₈ alkyl; and
X⁻ is a monovalent anion selected from the group consisting of F⁻, Cl⁻, Br⁻, I⁻, HO⁻, BF₄⁻, PF₆⁻, HCO₃⁻, NO₃⁻, and CH₃CO₂⁻, and
wherein the falling ball viscosity of 1 wt % polymer solution in dimethyl sulfoxide (DMSO) is from about 7 cP to about 30 cP.

In some embodiments, R₁, R₂, R₃, and R₄ are the same C₁₋₈ alkyl. In some embodiments, R₁, R₂, R₃, and R₄ are the same C₂₋₈ alkyl. In some embodiments, R₁, R₂, R₃, and R₄ are the same C₁₋₆ alkyl. In some embodiments, R₁, R₂, R₃, and R₄ are the same C₂₋₆ alkyl. In some embodiments, R₁, R₂, R₃, and R₄ are the same C₁₋₄ alkyl. In some embodiments, R₁, R₂, R₃, and R₄ are the same C₂₋₄ alkyl. In some embodiments, R₁, R₂, R₃, and R₄ are the same C₄ alkyl. In some embodiments, R₁, R₂, R₃, and R₄ are the same C₂ alkyl. In some embodiments, R₁, R₂, R₃, and R₄ are the same C₁ alkyl.

In some embodiments, any two of R₁, R₂, R₃, and R₄ are the same. In some embodiments, any three of R₁, R₂, R₃, and R₄ are the same. In some embodiments, any two of R₁, R₂, R₃, and R₄ are different from each other. In some embodiments, any three of R₁, R₂, R₃, and R₄ are different from each other. In some embodiments, all four of R₁, R₂, R₃, and R₄ are different from each other.

In some embodiments, R₁ is different from R₂, and R₃ is different from R₄.

In some embodiments, one or more of R₁ and R₂ is methyl, and one or more of R₃ and R₄ is methyl.

In some embodiments, one of R₁ and R₂ is methyl, and one of R₃ and R₄ is methyl. In some embodiments, R₁ is methyl and R₄ is methyl.

In some embodiments, one or more of R₁ and R₂ is C₂₋₆ alkyl, and one or more of R₃ and R₄ is C₂₋₆ alkyl. In some embodiments, one of R₁ and R₂ is C₂₋₆ alkyl, and one of R₃ and R₄ is C₂₋₆ alkyl. In some embodiments, R₂ is C₂₋₆ alkyl and R₃ is C₂₋₆ alkyl.

In some embodiments, one or more of R₁ and R₂ is C₄ alkyl, and one or more of R₃ and R₄ is C₄ alkyl. In some embodiments, one of R₁ and R₂ is C₄ alkyl, and one of R₃ and R₄ is C₄ alkyl. In some embodiments, one of R₁ and R₂ is n-butyl, and one of R₃ and R₄ is n-butyl. In some embodiments, R₂ is n-butyl and R₃ is n-butyl.

In some embodiments, one of R₁ and R₂ is methyl and the other one of R₁ and R₂ is C₂₋₆ alkyl. In some embodiments, one of R₃ and R₄ is methyl and the other one of R₃ and R₄ is C₂₋₆ alkyl. In some embodiments, one of R₁ and R₂ is methyl and the other one of R₁ and R₂ is n-butyl. In some embodiments, one of R₃ and R₄ is methyl and the other one of R₃ and R₄ is n-butyl. In some embodiments, R₁ is methyl, R₂ is butyl, R₃ is butyl, and R₄ is methyl.

In some embodiments, the repeating unit is Formula (II):

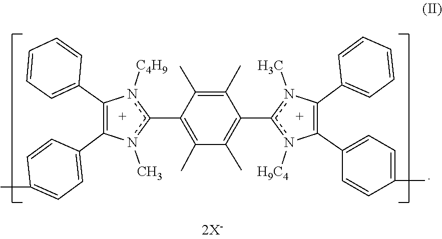

In some embodiments, X⁻ is selected from the group consisting of halide, hydroxide, alkoxide, tetrahalo borate, hexahalo phosphate, bicarbonate, nitrate, and carboxylate. In some embodiments, X⁻ is selected from the group consisting of F⁻, Cl⁻, Br⁻, I⁻, HO⁻, BF₄⁻, PF₆⁻, HCO₃⁻, NO₃⁻, and CH₃CO₂⁻. In some embodiments, X⁻ is Cl⁻. In some embodiments, X⁻ is HO⁻.

In some embodiments, the repeating unit is Formula (III):

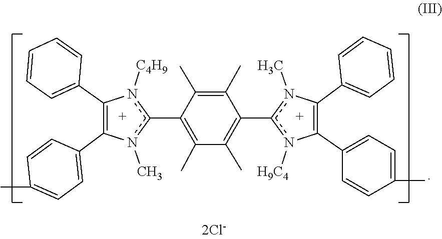

In some embodiments, substantially all R₁, R₂, R₃, and R₄ are alkylated. As used herein, “substantially all” means about 85% to about 100%, about 90% to about 100%, about 95% to about 100%, or about 98% to about 100% of all of R₁, R₂, R₃, and R₄ are alkylated.

In some embodiments, the tensile strength of the polymer is from about 50 MPa to about 100 MPa. In some embodiments, the tensile strength of the polymer is from about 50 MPa to about 75 MPa. In some embodiments, the tensile strength of the polymer is from about 75 MPa to about 100 MPa. In some embodiments, the tensile strength of the polymer is from about 60 MPa to about 90 MPa. In some embodiments, the tensile strength of the polymer is from about 70 MPa to about 90 MPa. In some embodiments, the tensile strength of the polymer is from about 60 MPa to about 80 MPa. Tensile strength is a maximum load that the polymer can support without fracture when being stretched, divided by the original cross-sectional area of the polymer.

In some embodiments, the falling ball viscosity of 1 wt % polymer solution in DMSO is from about 10 cP to about 30 cP. In some embodiments, the falling ball viscosity of 1 wt % polymer solution in DMSO is from about 7 cP to about 20 cP. In some embodiments, the falling ball viscosity of 1 wt % polymer solution in DMSO is from about 18 cP to about 30 cP. In some embodiments, the falling ball viscosity of 1 wt % polymer solution in DMSO is from about 18 cP to about 24 cP. Falling ball viscosity is a measurement of the viscosity of the polymer based on the time required for a spherical ball to fall a certain distance under gravity through a tube filled with said polymer.

In some embodiments, the molecular weight of the polymer is between about 500 and about 2000 kDa.

In another aspect, featured herein is a method of making the polymers as described and illustrated herein. In one embodiment, the method of making a polymer comprises forming a monomeric base unit of Formula (IV); alkylating a monomeric base unit nitrogen to form a dialkylated base unit; forming Ni(COD)₂ (Bis(cyclooctadiene)nickel(0)) in situ for use in polymerizing the dialkylated base unit; and polymerizing the dialkylated base unit to form a polymerized dialkylated base unit. In some embodiments, a base unit nitrogen of the polymerized dialkylated base unit is alkylated to form a tetraalkylated polymerized base unit comprising a repeating unit of Formula (I).

In some embodiments, Formula (IV) is:

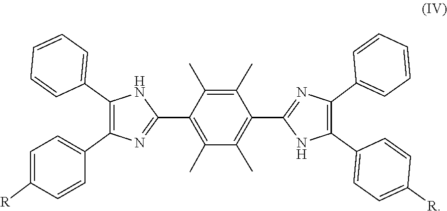

In some embodiments, R is Cl. In some embodiments, R is Br. In some embodiments, R is I.

In another aspect, featured herein is a method of making the polymers as described and illustrated herein. In one embodiment, the method of making a polymer comprises forming a monomeric base unit of Formula (V); alkylating a monomeric base unit nitrogen to form a dialkylated base unit; forming Ni(COD)₂ (Bis(cyclooctadiene)nickel(0)) in situ for use in polymerizing the dialkylated base unit; and polymerizing the dialkylated base unit to form a polymerized dialkylated base unit. In some embodiments, a base unit nitrogen of the polymerized dialkylated base unit is alkylated to form a tetraalkylated polymerized base unit comprising a repeating unit of Formula (I).

In some embodiments, Formula (V) is:

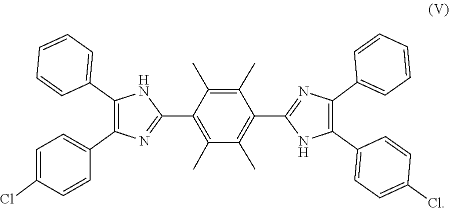 [SMILES: Cc1c(C)c(-c2nc(-c3ccccc3)c(-c3ccc(Cl)cc3)[nH]2)c(C)c(C)c1-c1nc(-c2ccc(Cl)cc2)c(-c2ccccc2)[nH]1]

In some embodiments, the polymers described herein can be made with Yamamoto coupling, as known to one of skill in the art. In a Yamamoto coupling, a monomeric unit comprising an aromatic halogen is coupled with another monomeric unit comprising an aromatic halogen, catalyzed by a Ni⁰ complex, to form an aryl-aryl covalent bond. A general Yamamoto coupling reaction follows: 2 ArCl→Ar—Ar. As another example, Yamamoto coupling can result in a polymer such as in the general Yamamoto coupling reaction: (ClAr—Y—ArCl)ₙ→ClAr—Y—Ar—(Ar—Y—Ar)ₙ₋₂—Ar—Y—ArCl.

Synthesis and characterization of the poly(bis-arylimidazolium) polymers of the present disclosure are described in Examples 1-6 below.

In an embodiment, featured herein is an ionic membrane comprising any polymer disclosed herein. In some embodiments, the ionic membrane is an AEM. In some embodiments, the ionic membrane is incorporated into a catalyst layer of a fuel cell, of an electrolyzer, of a redox flow battery, or of another electrochemical device.

In an embodiment, featured herein is an electrochemical device comprising any polymer disclosed herein, wherein the electrochemical device is a fuel cell, an electrolyzer, a redox flow battery, or another electrochemical device.

In an embodiment, featured herein is an electrochemical device comprising the ionic membrane as described herein, wherein the ionic membrane comprises a polymer disclosed herein, wherein the electrochemical device is a fuel cell, an electrolyzer, a redox flow battery, or another electrochemical device.

Polymer membranes derived from a poly(bis-arylimidazolium) as described herein exhibit suitable membrane flexibility and physical characteristics desirable for use as an anion-exchange membrane (AEM).

### EXAMPLES

Bis-arylimidazolium monomers with specified alkyl side chains produce robust, sterically protected poly(arylimidazolium) hydroxide anion exchange polymers that possess a combination of high ion-exchange capacity and exceptional stability under highly caustic conditions.

### Example 1

### Polymer Synthesis

Various monomers used in synthesis of polymers were prepared and characterized.

Synthesis of various poly(bis-arylimidazoliums) was accomplished via Yamamoto-coupling homo-polymerization of dichloro-imidazole monomers through two approaches: (1) ex situ Ni(COD)₂ (Synthesis I), and (2) in situ generation of Ni(COD)₂ (Synthesis II), to generate polymers having Structure (I).

### Synthesis I—Polymerization via Ex Situ Ni(COD)₂

Tetramethylated poly(bis-arylimidazolium) (TM-PBAI) was prepared by placing 2,2′-bipyridine (0.075 g, 0.48 mmol) in a 50 mL round-bottom flask, and the reaction flask evacuated and refilled with argon. Ni(COD)₂ (0.132 g, 0.48 mmol, Aldrich®) was transferred into the reaction mixture, and the flask was evacuated and purged with argon repeatedly. Anhydrous DMF (5 mL) was added, and the mixture was heated to 80° C. for 30 minutes. In a separate flask, 2,2′-(2,3,5,6-tetramethyl-2-yl)bis(3-methyl-4-chlorophenyl-5-diphenyl-imidazole) (0.1335 g, 0.2 mmol) and 5 mL anhydrous DMF was added. The flask was purged with argon, and after the catalyst was heated for 30 minutes, the monomer solution was transferred into the catalyst solution. The resulting mixture was heated at 80° C. while stirring for 20 h. After cooling, the solution was poured into 200 mL of 6 M HCl, to consume the catalyst. The solid was filtered and washed with water, aqueous sodium bicarbonate, and acetone. After drying in vacuo, the solid was dissolved in 5 mL DCM and 5 mL DMSO. 20 equivalents of MeI was added, and the solution heated to 80° C. for three days. The polymer was precipitated in 100 mL ethyl acetate, washed with acetone, and filtered to yield a brown solid 0.1695 g (100% yield).

The polymers obtained following the aforementioned method had a medium range molecular weight (MW=140 kDa, Polydispersity Index=1.70).

The polymer having Structure (VI) was obtained by the foregoing method:

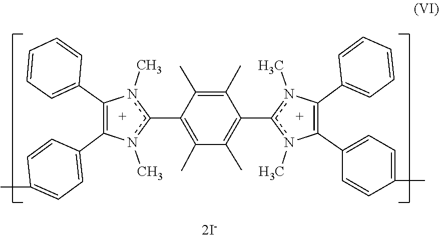

### Synthesis II—Polymerization via In Situ Ni(COD)₂

Synthesis of various poly(bis-arylimidazoliums) has been accomplished via Yamamoto-coupling homo-polymerization of dichloro-imidazole monomers to obtain high molecular weight (MW=500-2000 kDa, Polydispersity Index=2-4) poly(bis-arylimidazoles) using in-situ generated bis(cyclooctadiene)nickel(0) (Ni(COD)₂).

A 5-L round-bottom flask equipped with a rubber septum was charged, under a stream of argon (or nitrogen), with anhydrous Ni(acac)₂ (CAS 3264-82-2, 154.2 g) and 1,5-cyclooctadiene (CAS 111-78-4, 380 mL). This solution was stirred and cooled to 0° C., upon which 1.0 M diisobutylaluminum hydride (DIBAL-H) in hexanes (CAS 1191-15-7, 1.21 L) was added slowly, keeping the temperature at 0° C. during the addition. After the addition was complete, the stirring of the brownish-yellow solution was allowed to continue for 2 h at 0° C. During this time period, yellow-orange crystals of Ni(COD)₂ were observed to precipitate. The stirring was stopped and the crystals allowed to settle. Keeping the flask as close to 0° C. as possible, the solution was carefully decanted under argon or nitrogen to remove it while leaving the crystals of Ni(COD)₂ behind. Once the solution was removed, the crystals were washed twice with cold (2-5° C.) anhydrous diethyl ether (CAS 60-29-7, 0.6 L each time) or anhydrous hexanes (CAS 110-54-3), and the solution removed again by decantation.

To the crystals were added 2,2′-bipyridine (CAS 366-18-7, 100 g) and 2,2′-(2,3,5,6-tetramethyl-2-yl)bis(3-methyl-4-chlorophenyl-5-diphenyl-imidazole) (80 g). To this mixture, 2.6 L of anhydrous DMF (CAS 68-12-2) was added by syringe or cannula through the septum, and the mixture was heated at 60° C. for 2 h after which the original purple color of the solution had diminished. Argon (or nitrogen) purging was terminated but the flask remained sealed.

To accomplish quarternization of imidazole polymer, alkyl iodide (such as 1-iodobutane, CAS 542-69-8, 350 g) was added into the reaction flask. Stirring of the reaction mixture was maintained at 110° C. for 18 h. The reaction mixture was cooled down to 40-50° C., and 0.24 L of concentrated HCl (˜36%) was added gradually (to prevent precipitation of polymer at this stage). The mixture was stirred until all the black “Ni” was reacted and the solution turned into a clear greenish blue color. The polymer was then precipitated in 13 L of water. The off-white product was filtered, and washed with water until the filtrate pH was at 7.0. The polymer was collected and dried in an oven overnight at 100° C.

The degree of alkylation in polymers was determined by integration of representative peaks of said polymer of (a) R₁, R₂, R₃, R₄=Me, wherein the degree of functionality was calculated based on the ratio between N—CH₃ protons out of possible 12 (δ 3-4 ppm) when aromatic protons are fixed at 18 (δ 7.0-8.1 ppm), and (b) R₁, R₂, R₃, R₄=a combination of two methyl and two butyl groups, wherein the degree of functionality was calculated based on the ratio between butyl CH₃ protons out of possible 6 (δ 0.5-0.7 ppm) when aromatic protons were fixed at 18 (δ 7.0-8.1 ppm).

### Example 2

### Ion-Exchange Capacity

Ion-exchange capacity (IEC) for various (poly(bisarylimidazolium) polymers was determined via the back titration method:

To a sample of polymer (0.1 g) in an Erlenmeyer flask (250 mL), HCl (25 mL of 0.0100 M) was added, and the polymer soaked for 2 h. The amount of HCl volume was varied, and it depended on degree of alkylation of the polymer. 1 wt % of phenolphthalein as indicator (in ethanol) (10 drops) was added into the Erlenmeyer flask.

The polymer solution in HCl, with phenolphthalein indicator, was titrated with 0.0100 M NaOH until the indicator changed colour (little pink color), indicating the end point of the titration.

IEC value of the polymer was calculated using the following equation:

$$I E C ( mmol / g ) = [ ( {V}_{HC 1} \times {M}_{HC 1} ) - ( {V}_{NaOH} \times {M}_{NaOH} ) ] / {W}_{dry}$$

where V_{HCl} and M_{HCl}, as well as V_{NaOH} and M_{NaOH}, are the volume and concentration of the HCl and NaOH solutions, respectively, and W_{dry} is the weight of the dry polymer used for the titration experiment (0.100 g in this example).

TABLE 1
Examples of Poly(bis-arylimidazoliums) and corresponding IEC

| | | Degree of | IEC |
|---|---|---|---|
| | Polymer | Functionality (%) | (mmol/g) |
| 1 | R₁, R₃ = Me; R₂, R₄ = Me | 89-92 | 2.3-2.4 |
| 2 | R₁, R₃ = Me; R₂, R₄ = Bu | 91-99 | 2.2-2.5 |
| 3 | R₁, R₃ = Me; R₃, R₄ = Et | 90-95 | 2.3-2.5 |

Ion-exchange capacity (IEC) for AEM was determined via chloride counter ion concentration by titration (Mohr's Method):

To a sample of poly(bis-arylimidazolium) (0.1 g) in an Erlenmeyer flask was added 100 mL deionized water, and the polymer was allowed to soak for a couple of hours. Potassium chromate (1 mL, 0.25 M) as indicator was added into the polymer solution, and the solution titrated with silver nitrate solution (0.1 M). Although the silver chloride that forms is a white precipitate, the chromate indicator initially gives the cloudy solution a faint lemon-yellow color. The end point is the formation of brown silver chromate.

The concentration of chloride ions in mmol/g was calculated by mmoles of silver nitrate consumed, and the moles of silver nitrate reacting was calculated.

The following reaction equation was used to determine the moles of chloride ions reacting:

Ag⁺_{(aq)}+Cl⁻_{(aq)}→AgCl₍ₛ₎.

TABLE 2
Examples of Poly(bis-arylimidazoliums) and
corresponding IECs by Mohr Method

| | | Degree of | IEC |
|---|---|---|---|
| | Polymer | Functionality (%) | (mmol/g) |
| 1 | R₁, R₂ = Me; R₃, R₄ = Me | 89 | 2.1 |
| 2 | R₁, R₂ = Me R₃, R₄ = Bu | 91 | 1.9 |
| 3 | R₁, R₂ = Me R₃, R₄ = Et | 82 | 1.9 |

### Example 3

### Cl⁻ Ion Conductivity of Membranes

The polymer membranes were prepared by solution casting a polymer solution (7-10 wt % in a solvent e.g., DMF, DMSO, etc.) using a Doctor Blading casting table. The membranes were dried in a conventional oven at 80° C. for one hour to produce a membrane with thickness around 50 microns. In-plane chloride ion conductivities of partially and fully hydrated (or water soaked wet) membranes were determined by AC impedance spectroscopy using a Solartron SI 1260 impedance/gain-phase analyzer at 25° C.

The membranes were pressed onto two platinum electrodes by two Teflon blocks. Impedance measurements were performed using 100 mV sinusoidal AC voltage between 10 MHz and 100 MHz. The resistance (R, Ω) of the membrane was determined by fitting a standard Randles equivalent circuit to the obtained Nyquist plot. By using the obtained resistance, the dimensions of the membrane at the given conditions, and distance between the platinum electrodes (d, cm), the Cl⁻ conductivity (σ, mS/cm) was calculated using the equation shown below:

$${σ}_{Cl -} = \frac{d}{width \times thickness \times R}$$

TABLE 3
Examples of Poly(bis-arylimidazoliums) and
their conductivities (σ)

| | | Degree of | σ_{Cl-}, |
|---|---|---|---|
| | Polymer | Functionality (%) | mS/cm |
| 1 | R₁, R₂ = Me; R₃, R₄ = Me | 89-90 | 7-9 |
| 2 | R₁, R₃ = Me R₂, R₄ = Bu | 91-98 | 6-8 |
| 3 | R₁, R₃ = Me R₂, R₄ = Et | 82 | 5 |

Table 3 data shows that a higher degree of alkylation results in higher ionic (chloride) conductivity.

### Example 4

### Stability of Poly(bis-arylimidazolium) Membranes

A membrane of a polymer of Structure (I) is cast from certain solvents such as DMF or DMSO, or a combination of low boiling point (BP) solvents such as methanol and acetone, at a typical 7 wt %. The cast membrane was dried in an oven for a period of 1 to 2 hours, depending on the BP of solvent used. After drying, the membrane with a typical thickness of 50 microns was cut into small pieces (5×5 cm). Membrane pieces were soaked in concentrated alkali solution (such as KOH 1-5 M) for an extended period of time (up to 4 weeks) and subjected to drying, tensile and conductivity tests.

TABLE 4
Examples of Poly(bis-arylimidazoliums) Membrane
Tensile Strength and Conductivity (σ)
after soaking in KOH (1M) for 4 weeks

| | | Viscosity | Tensile, | σ, |
|---|---|---|---|---|
| | Polymer | (cP) | MPa (Strain %) | mS/cm |
| 1 | R₁, R₂ = Me; R₃, R₄ = Me | 16.8 | 30 (25%) | 10 |
| 2 | R₁, R₃ = Me R₂, R₄ = Bu | 16.0 | 50 (26%) | 8.9 |
| 3 | R₁, R₃ = Me R₂, R₄ = Et | 15.1 | NA | NA |

### Example 5

### Tensile Properties

The tensile strength of a polymer correlates with the degree of polymerization (or molecular weight) of a polymer. The relationship between strength and the degree of polymerization can also been expressed quantitatively, as shown in Equation 1:

$${T}_{s} = {T}_{g ∞} - \frac{C}{D {P}_{n}} \qquad Equation l$$

wherein Tₛ is the tensile strength for the degree of polymerization for the polymer (DPₙ), T_{g∞} is equal to the extrapolated tensile strength for an infinite DP, and C is equal to a constant characteristic for a given polymer.

Membrane test samples were cut using a standard ASTM D638-4 die-cutter into a standard dumbbell sample (width is 0.6 cm and length is 3.6 cm), or a non-standard strip sample (width is 1 cm and length is 5 cm). The tensile Stress (MPa), Strain (% elongation at break) and Elastic Modulus (MPa) were measured using an Instron 3345 Tensile Tester using a crosshead speed of 5 mm min⁻¹. The error reported is the standard deviation (Std.D) of four measurements. Typical test results are summarized in Table 5.

TABLE 5
Tensile test results of Poly(bis-arylimidazolium) membranes

| | | | | | | | Elastic | |
|---|---|---|---|---|---|---|---|---|
| | | Viscosity | Stress | Std. | Strain | Std. | Modulus | Std. |
| | Polymer | (cP) | (MPa) | D_1 | (%) | D_2 | (MPa) | D |
| 1 | R₁, R₂ = Me; | 8.5 | 67.9 | 13.8 | 25.6 | 13.6 | 1316.5 | 28.1 |
| | R₃, R₄ = Me | | | | | | | |
| 2 | R₁, R₂ = Me; | 12.0 | 75.7 | 3 | 54.6 | 2.9 | 2369.5 | 62.9 |
| | R₃, R₄ = Me | | | | | | | |
| 3 | R₁, R₃ = Me | 22.7 | 100.5 | 4.5 | 31.7 | 4 | 1617.7 | 34.3 |
| | R₂, R₄ = Bu | | | | | | | |
| 4 | R₁, R₃ = Me | 18.8 | 87.2 | 5.2 | 43.5 | 4.9 | 1227.9 | 37.2 |
| | R₂, R₄ = Bu | | | | | | | |
| 5 | R₁, R₃ = Me | 23.8 | 83.3 | 3.3 | 63.1 | 3.6 | 1194.4 | 1.4 |
| | R₂, R₄ = Bu | | | | | | | |
| 6 | R₁, R₃ = Me | 29.0 | 82.8 | 3.7 | 50 | 4.3 | 1320.8 | 41.3 |
| | R₂, R₄ = Bu | | | | | | | |

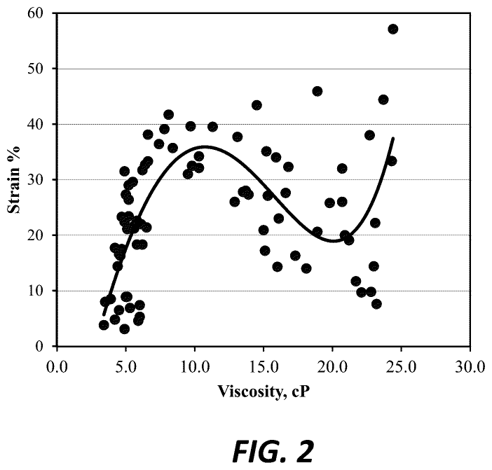

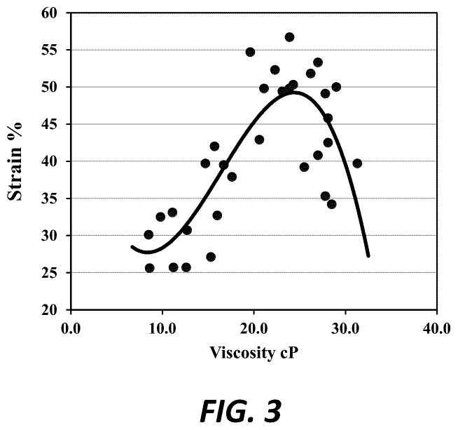

Data shown in Table 5 shows that membranes of polymers with viscosity between 10-30 cP have favorable tensile properties, namely, Stress of 60 to 100 MPa and Strain of 30% to 60%. This is further supported by the data shown in FIGS. 2 and 3, wherein polymers having a viscosity between 10-30 cP, corresponding to a molecular weight>500 kDa, when incorporated into membranes, demonstrate favorable mechanical properties such as strain (elongation at break) of about 30% to about 60%.

### Example 6

### Viscosity Test Using Gilmont™ GV-2200 Falling Ball Viscometer

The viscosity of polymer solutions was measured using falling ball viscometry. All polymer solutions were filtered through a 0.45 micrometer filter disk before viscosity measurement. A Glass Ball (GF-1332) was placed in the viscometer tube and the polymer solution was added until the tube was almost full. The tube was inverted upside down and the ball was allowed to slowly move and drop into its locknut. The tube was inverted again, and the ball released using the locknut. Care was taken to ensure that no bubbles were around or below the ball. The ball slowly moved down through the polymer solution. A stopwatch was used to measure the time it took for the ball to travel between the two sets of fiduciary lines.

Viscosity was calculated using the following equation:

$$Viscosity ( μ ) = K ( {d}_{ball} - {d}_{s o l v e n t} ) \times t / 60$$

wherein:
μ=viscosity in centipoises (cP)
d_{ball}=density of ball (g/mL) (2.53 for glass, 8.02 for stainless steel, and 16.6 for tantalum)
dₛₒₗᵥₑₙₜ=density of liquid (g/mL)
t=time of descent (seconds)
K=viscometer constant (measured using a known solvent)

TABLE 6
Examples of Polymers with GPC Mol. Wt. and Falling Ball

| | Polymer | GPC MW (kDa) | Viscosity, cP |
|---|---|---|---|
| 1 | R₁, R₃ = Me; R₂, R₄ = Me | 450-480 | 8.5-8.9 |
| 2 | R₁, R₃ = Me; R₂, R₄ = Bu | 500-2000 | 10-30 |
| 3 | R₁, R₃ = Me; R₂, R₄ = Me | 390-400 | 6.6-7.1 |
| 4 | R₁, R₃ = Me; R₂, R₄ = Me | 190-210 | 4.8-5.2 |

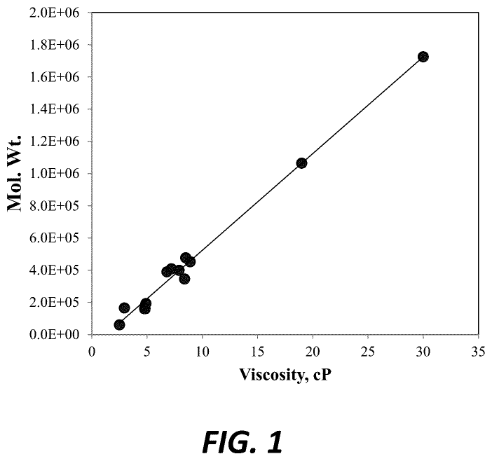

As can be seen from the Mol. Wt. vs. Viscosity plot in FIG. 1, a viscosity between 10-30 cP (Table 6) corresponds to a molecular weight of 500 to 2000 kDa.

### Example 7

### Molecular Weight

The molecular weight (MW) of the polymers was determined by a Gel Permeation Chromatography (GPC) System with the system composed of Waters® 1515 Isocratic HPLC pump, Waters® 2414 Refractive Index and Waters® 2487 Dual wavelength Absorbance Detectors, Column Shodex OHpak SB-806 and Column Heater Module CHM, and with a DMF+0.05 M LiBr mobile phase.

As can be seen from the Mol. Wt. vs. Viscosity plot in FIG. 1, a viscosity between 10-30 cP (Table 6) corresponds to a molecular weight of 500 to 2000 kDa.

Based on strain %, viscosity, and the MW of the polymers depicted in FIGS. 1-3, a preferred range for MW of the polymer is derived between 500 to 2000 kDa.

### Example 8

### Membrane Characteristics

Membranes that were cast from the preferred and selected range of viscosity were subjected to characterization by ex situ methods such as tensile, conductivity, and stability tests in alkali solutions in wet/dry cycles, as well as in situ methods, such as a water electrolysis device, to monitor real-time membrane performance.

The terminology used in the description of the invention herein is for the purpose of describing particular embodiments only and is not intended to be limiting of the invention. All publications, patent applications, patents and other references mentioned herein are incorporated by reference in their entirety. In case of a conflict in terminology, the present specification is controlling.

While illustrative embodiments have been illustrated and described, it will be appreciated that various changes can be made therein without departing from the spirit and scope of the invention.

### Embodiments

Embodiment 1: A polymer, comprising a repeating unit of Formula (I):

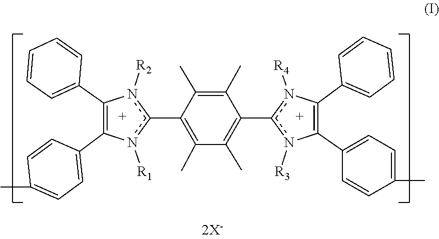

wherein:
R₁, R₂, R₃, and R₄ are each independently C₁₋₈ alkyl, and
wherein the falling ball viscosity of 1 wt % polymer solution in dimethyl sulfoxide (DMSO) is from about 7 cP to about 30 cP.

Embodiment 2: The polymer of Embodiment 1, wherein R₁, R₂, R₃, and R₄ are the same C₁₋₈ alkyl.

Embodiment 3: The polymer of Embodiment 1, wherein R₁ is different from R₂, and R₃ is different from R₄.

Embodiment 4: The polymer of any one of Embodiments 1-3, wherein one or more of R₁ and R₂ is methyl, and one or more of R₃ and R₄ is methyl.

Embodiment 5: The polymer of any one of Embodiments 1, 3, or 4, wherein one of R₁ and R₂ is methyl, and one of R₃ and R₄ is methyl.

Embodiment 6: The polymer of any one of Embodiments 1-3, wherein one or more of R₁ and R₂ is C₂₋₆ alkyl, and one or more of R₃ and R₄ is C₂₋₆ alkyl.

Embodiment 7: The polymer of any one of Embodiments 1, 3, or 6, wherein one of R₁ and R₂ is C₂₋₆ alkyl, and one of R₃ and R₄ is C₂₋₆ alkyl.

Embodiment 8: The polymer of any one of Embodiments 1 or 3-7, wherein one of R₁ and R₂ is butyl, and one of R₃ and R₄ is butyl.

Embodiment 9: The polymer of any one of Embodiments 1 or 3-8, wherein the repeating unit is Formula (II):

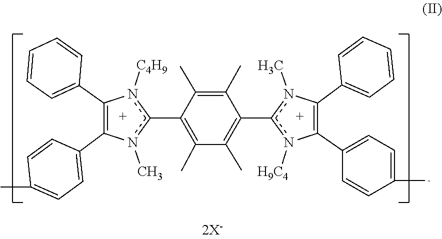

Embodiment 10: The polymer of any one of Embodiments 1-9, wherein X⁻ is selected from the group consisting of F⁻, Cl⁻, Br⁻, I⁻, HO⁻, BF₄⁻, PF₆⁻, HCO₃⁻, NO₃⁻, and CH₃CO₂⁻.

Embodiment 11: The polymer of any one of Embodiments 1-10, wherein X⁻ is Cl⁻.

Embodiment 12: The polymer of any one of Embodiments 1 or 3-11, wherein the repeating unit is Formula (III):

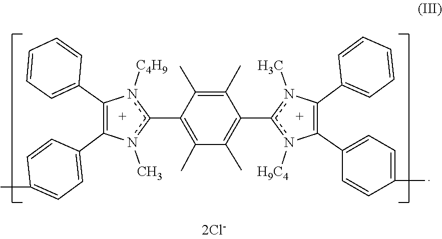

Embodiment 13: The polymer of any one of Embodiments 1-12, wherein the tensile strength of polymer membrane is from about 50 MPa to about 100 MPa.

Embodiment 14: The polymer of any one of Embodiments 1-13, wherein the falling ball viscosity of 1 wt % polymer solution in DMSO is from about 10 cP to about 30 cP.

Embodiment 15: The polymer of any one of Embodiments 1-14, wherein a molecular weight is between about 500 and about 2000 kDa.

Embodiment 16: A method of making a polymer, comprising:
forming a monomeric base unit of Formula (IV);
alkylating a monomeric base unit nitrogen to form a dialkylated base unit;
forming Ni(COD)₂ in situ for polymerizing the dialkylated base unit;
polymerizing the dialkylated base unit to form a polymerized dialkylated base unit; and
alkylating a polymerized dialkylated base unit nitrogen to form a tetraalkylated polymerized base unit comprising a repeating unit of Formula I,
wherein Formula (IV) is:

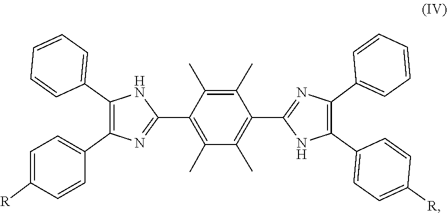

wherein R is Cl, Br, or I; and
wherein Formula (I) is:

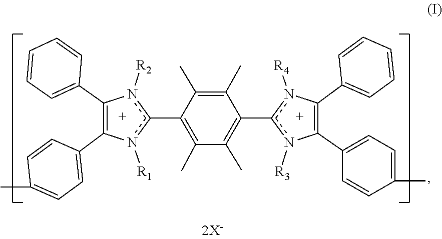

wherein:
R₁, R₂, R₃, and R₄ are each independently C₁₋₈ alkyl.

Embodiment 17: An ionic membrane comprising a polymer of any one of Embodiments 1-15.

Embodiment 18: The ionic membrane of Embodiment 17, incorporated into a catalyst layer of a fuel cell, of an electrolyzer, of a redox flow battery, or of another electrochemical device.

Embodiment 19: An electrochemical device comprising the polymer of any one of Embodiments 1-15, or of the ionic membrane of Embodiment 17, wherein the electrochemical device is a fuel cell. an electrolyzer, a redox flow battery, or another electrochemical device.

While illustrative embodiments have been illustrated and described, it will be appreciated that various changes can be made therein without departing from the spirit and scope of the invention.

The embodiments of the invention in which an exclusive property or privilege is claimed are defined as follows:

1. A polymer, comprising a repeating unit of Formula (I): 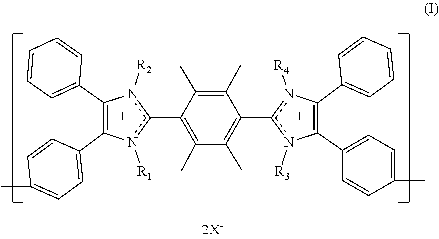
wherein:
R₁, R₂, R₃, and R₄ are each independently C₁₋₈ alkyl, and
wherein the falling ball viscosity of 1 wt % polymer solution in dimethyl sulfoxide (DMSO) is from about 7 cP to about 30 cP.

2. The polymer of claim 1, wherein R₁, R₂, R₃, and R₄ are the same C₁₋₈ alkyl.

3. The polymer of claim 1, wherein R₁ is different from R₂, and R₃ is different from R₄.

4. The polymer of claim 1, wherein one or more of R₁ and R₂ is methyl, and one or more of R₃ and R₄ is methyl.

5. The polymer of claim 1, wherein one of R₁ and R₂ is methyl, and one of R₃ and R₄ is methyl.

6. The polymer of claim 1, wherein one or more of R₁ and R₂ is C₂₋₆ alkyl, and one or more of R₃ and R₄ is C₂₋₆ alkyl.

7. The polymer of claim 1, wherein one of R₁ and R₂ is C₂₋₆ alkyl, and one of R₃ and R₄ is C₂₋₆ alkyl.

8. The polymer of claim 1, wherein one of R₁ and R₂ is butyl, and one of R₃ and R₄ is butyl.

9. The polymer of claim 1, wherein the repeating unit is Formula (II):

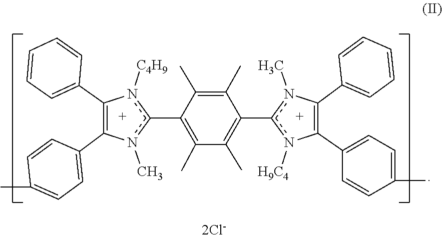

10. The polymer of claim 1, wherein X⁻ is selected from the group consisting of F⁻, Cl⁻, Br⁻, I⁻, HO⁻, BF₄⁻, PF₆⁻, HCO₃⁻, NO₃⁻, and CH₃CO₂⁻.

11. The polymer of claim 1, wherein X⁻ is Cl⁻.

12. The polymer of claim 1, wherein the repeating unit is Formula (III):

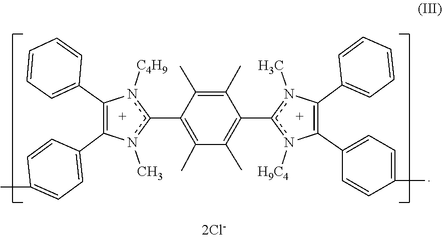

13. The polymer of claim 1, wherein the tensile strength of polymer membrane is from about 50 MPa to about 100 MPa.

14. The polymer of claim 1, wherein the falling ball viscosity of 1 wt % polymer solution in DMSO is from about 10 cP to about 30 cP.

15. The polymer of claim 1, wherein a molecular weight is between about 500 and about 2000 kDa.

16. A method of making a polymer, comprising:
forming a monomeric base unit of Formula (IV);
alkylating a monomeric base unit nitrogen to form a dialkylated base unit;
forming Ni(COD)₂ in situ for polymerizing the dialkylated base unit;
polymerizing the dialkylated base unit to form a polymerized dialkylated base unit; and
alkylating a polymerized dialkylated base unit nitrogen to form a tetraalkylated polymerized base unit comprising a repeating unit of Formula I,
wherein Formula (IV) is: 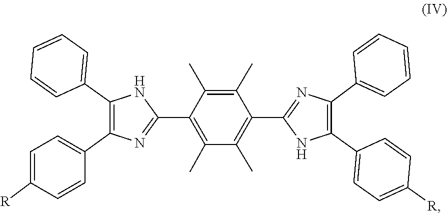
wherein R is Cl, Br, or I; and
wherein Formula (I) is: 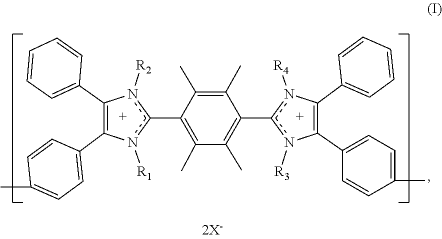
wherein:
R₁, R₂, R₃, and R₄ are each independently C₁₋₈ alkyl.

17. An ionic membrane comprising a polymer of claim 1.

18. The ionic membrane of claim 17, incorporated into a catalyst layer of a fuel cell, of an electrolyzer, of a redox flow battery, or of another electrochemical device.

19. An electrochemical device comprising the polymer of claim 1, wherein the electrochemical device is a fuel cell, an electrolyzer, a redox flow battery, or another electrochemical device.
